(19) United States
(12) Patent Application Publication
(10) Pub. No.: US 2007/0004737 A1
(43) Pub. Date: Jan. 4, 2007

(54) Process of purifying tadalafil

(76) Inventor: Inbal Ornan, Arad (IL)

Correspondence Address: KENYON & KENYON LLP, ONE BROADWAY, NEW YORK, NY, 10004, US

(21) Appl. No.: 11/364,599

(22) Filed: Feb. 27, 2006

Related U.S. Application Data
(60) Provisional application No. 60/656,664, filed on Feb. 25, 2005.
(60) Provisional application No. 60/683,058, filed on May 19, 2005.
(60) Provisional application No. 60/736,807, filed on Nov. 14, 2005.
(60) Provisional application No. 60/737,080, filed on Nov. 15, 2005.
(60) Provisional application No. 60/677,514, filed on May 3, 2005.

(51) Int. Cl. A61K 31/498; C07D 487/14

(52) U.S. Cl. 514/249; 544/343

(57) ABSTRACT

The invention is directed to a method of purifying tadalafil by crystallization of tadalafil from a solution of crude tadalafil in a suitable crystallization solvent.

### RELATED APPLICATIONS

This application claims the benefit of U.S. Provisional Application No. 60/656,664, filed Feb. 25, 2005; U.S. Provisional Application No. 60/683,058, filed May 19, 2005; U.S. Provisional Application No. 60/736,807, filed Nov. 14, 2005; and U.S. Provisional Application No. 60/737,080, filed Nov. 15, 2005 and U.S. Provisional Application No. 60/677,514, filed May 3, 2005. The contents of these applications are incorporated herein by reference.

### FIELD OF THE INVENTION

The invention is directed to processes of purifying tadalafil by crystallization.

### BACKGROUND OF THE INVENTION

Tadalafil, (6R-trans)-6-(1,3-benzodioxol-5-yl)-2,3,6,7,12,12a-hexahydro-2-methyl-pyrazino[1′, 2′:1,6]pyrido[3,4-b]indole-1,4-dione, with the structural formula shown below, is a white crystalline powder. (CAS# 171596-29-5). Tadalafil is a potent and selective inhibitor of the cyclic guanosine monophosphate (cGMP)—specific phosphodiesterase enzyme, PDE5. The inhibition of PDE5 increases the amount of cGMP, resulting in smooth muscle relaxation and increased blood flow. Tadalafil is therefore currently used in the treatment of male erectile dysfunction, and is commercially available as CIALIS®.

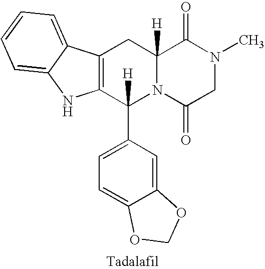

### Tadalafil

U.S. Pat. No. 5,859,006 describes the synthesis of tadalafil via the cyclization of TDCL (i.e., cis-methyl 1,2,3,4-tetrahydro-2-chloroacetyl-1-(3,4-methylenedioxyphenyl)-9H-pyrido[3,4-b]indole-3-carboxylate) using methylamine by purification by flash chromatography, followed by subsequent crystallization from methanol. Crude tadalafil typically requires additional purification steps, such as multiple extractions, crystallization, and/or flash chromatography, to remove the impurities present in the compound after synthesis is complete. Such purification processes increase the cost of producing tadalafil. Also, when repeating the US '006 process, about 250 volumes of methanol were necessary for the crystallization step.

Inexpensive and simple methods of purifying crude tadalafil are desirable to allow the preparation of tadalafil in a cost efficient manner.

### SUMMARY OF THE INVENTION

The present invention provides a process for the purification of tadalafil that includes the step of crystallizing crude tadalafil from a solution of tadalafil in a solvent selected from the group consisting of C₂-C₆ aliphatic alcohols and mixtures of ketones or nitrites with a hydroxylic solvent.

The tadalafil obtained by the above process is free of contaminates, and preferably has an assay of about 100% w/w as measured by HPLC.

### DETAILED DESCRIPTION OF THE INVENTION

The invention provides a process of purifying tadalafil that includes the step of crystallizing tadalafil using a suitable crystallization solvent. This process comprises providing a solution of crude tadalafil in a suitable crystallization solvent, and crystallizing purified tadalafil from the solution. The process of the present invention effects a purification of crude tadalafil. In particular, the level of methyl amine, as the free base or as the hydrochloride, is reduced. For example, when the level of methyl amine in crude tadalafil is about 2% to 10%, the level of methyl amine, as free base or hydrochloride, in the tadalafil obtained from the method of the present invention will be about 20 ppm to about 300 ppm, as may be determined by one skilled in the art using conventional methods of analysis.

The process of the invention includes the step of crystallizing crude tadalafil from about 15 to about 100 volumes of a suitable crystallization solvent (i.e, 250 mL of crystallization solvent per 1 gram of tadalafil starting material).

“Crude tadalafil” refers to tadalafil that is used as a starting material in the process of the invention. Crude tadalafil typically contains about 10% impurities, in particular the impurity methyl amine or its hydrochloride salt. Crude tadalafil can be obtained from any source or process known in the art; for example, the tadalafil may be obtained by the process disclosed in U.S. Pat. No. 5,859,006 or the process disclosed in co-pending U.S. application Ser. No. 60/656,664. application Ser. No. 60/656,664 discloses the preparation of tadalafil in a minimum of process steps by reacting the intermediate TDCL with methylamine under particular reaction conditions.

As used herein, the terms “pure tadalafil” and “purified tadalafil” refer to tadalafil having about 0.5% area by HPLC or less impurities.

Preferably, tadalafil obtained from the purification process of the invention has about 0.2% or less impurities as measured by HPLC.

As used herein, the term “free of contaminates”, in reference to tadalafil, refers to tadalafil that has an assay of at least about 99.5% w/w. As such, tadalafil that is free of contaminates does not contain either organic or inorganic impurities.

As used herein, “suitable crystallization solvent” refers to a solvent in which crude tadalafil is soluble to the extent of at least about 1 gram in 100 mL. Preferred crystallization solvents useful in the method of the invention include aliphatic alcohols and mixtures of at least one ketone or nitrile with a hydroxylic solvent.

Preferably, the crystallization solvent is selected from the group consisting of: C₂-C₆ aliphatic alcohols and mixtures of ketones or nitriles with a hydroxylic solvent. More preferably, the solvent is selected from the group consisting of: butanol, a mixture of acetone and methanol, and mixtures of acetone or acetonitrile with water. Most preferably, the solvent is a mixture of water and acetone.

When the solvent is a mixture of acetone and water, tadalafil Form I, as analyzed by XRD, is obtained from the purification process. Form I is described in U.S. application Ser. No. 11/265,880, and is characterized by an x-ray diffraction pattern with characteristic reflections at about 7.3, 10.6, 12.6, 14.6, 18.5, 21.8 and 24.3±0.2° 2θ.

As used herein, the term “ketone” refers to an water-miscible organic compound having the general formula R₁(CO)R₂, wherein each R is a linear or branched alkyl group having from one to about 4 carbon atoms. Preferred ketones for use in the process of the present invention are acetone, methyl ethyl ketone and methyl isobutyl ketone. A most preferred ketone is acetone.

As used herein, the term “nitrile” refers to an organic compound having a —CN functional group. Aliphatic nitrites are preferred. Acetonitrile is a particularly preferred nitrile for use in the process of the invention.

As used herein, the term “aliphatic alcohols” refers to aliphatic alcohols having the general formula Cₙ(H₂)ₙ₊₁OH, wherein n is from at least about 2 to about 6. Preferred aliphatic alcohols for use in the practice of this and other embodiments of the present invention are ethanol, 1-propanol, isopropanol and n-butanol. n-butanol is a most preferred aliphatic alcohol.

As used herein, the term “hydroxylic solvent” refers to compounds, liquid at room temperature, having the formula R—OH, wherein R is H or a linear or branched alkyl group having up to about 3 carbon atoms. Water and methanol are useful hydroxylic solvents in the practice of the invention.

The amount of crystallization solvent used in the process of the invention is an amount sufficient to substantially dissolve the amount of tadalafil starting material. By “substantially dissolve” is meant that at least 50% of the tadalafil starting material is dissolved in the solution at about 25° C. to about 28° C. The amount of recrystallization solvent used in the recrystallization process of the present invention depends on, among other things, the scale of the reaction and the solubility of crude tadalafil in the particular organic solvent. Typically, about 15 to about 100 volumes of recrystallization solvent per gram of tadalafil are used in the process of the invention. Preferably, the amount of recrystallization solvent is about 20 to about 70 volumes per gram of crude tadalafil.

The process of the invention includes the step of providing a solution of crude tadalafil in a crystallization solvent. In one embodiment, the solution is provided by combining crude tadalafil and a desired amount of crystallization solvent and, preferably, heating the combination to obtain a solution of crude tadalafil in suitable crystallization solvent. The solution is preferably heated for a time sufficient to obtain a clear solution. The time required to obtain a clear solution is typically about 30 minutes to six hours, and preferably is about one hour to about three hours.

The solution is heated to a temperature of about 25° C. to about 140° C., and preferably is heated to a temperature of about 50° C. to about 125° C. More preferably, the solution is heated to a temperature of about 50° C. to about 95° C. Heating can be performed at a pressure of about 1 atmosphere (760 mmHg) to about 6 atmospheres. The solution is preferably stirred during heating. Optionally, the volume of the solution may be reduced by distillation.

Crystallization of purified tadalafil from the solution can be achieved by any means known in the art, including precipitation and distillation. Preferably, the solution of tadalafil and crystallization solvent is cooled to a temperature of about −20° C. to about 90° C. to induce crystallization of purified tadalafil. More preferably, the solution is cooled to a temperature of about 10° C. to about 30° C. Cooling of the solution can be performed continuously, in one step, or it can be performed in more than one step. For example, the solution can be cooled first to one temperature and subsequently cooled to a second temperature.

Crystallization of tadalafil from the solution can include the step of seeding the solution. Seeding is preferably performed during cooling of the solution. Seeding can be performed by any method known in the art, such as by adding a crystal to the solution or scratching the side of a glass reactor containing the solution of tadalafil.

The process of the invention optionally comprises the steps of filtering and washing the solution after the crystallization of purified tadalafil. Purified tadalafil can be washed with at least one volume of water, methanol, acetone, or butanol.

Having described the invention with reference to certain preferred embodiments, other embodiments will become apparent to one skilled in the art from consideration of the specification. The invention is further defined by reference to the following nonlimiting examples describing in detail the process of the invention in certain of its embodiments.

### EXAMPLES

### Example 1

### Crystallization of Tadalafil in n-Butanol

Crude tadalafil (30 g of dry base) and butanol (2400 mL) were combined to form a solution of crude tadalafil in a 3 liter reactor equipped with a mechanical stirrer, condenser, and thermometer. The solution was heated to 125° C. and stirred at a rate of 100 rpm for about one hour to obtain a clear solution. The solution was cooled to 90° C. and seeding was performed. The mixture was stirred at about 90° C. for one hour and then cooled to 10° C. over about 12 hours. The mixture was stirred at about 10° C. for an additional three hours, filtered under vacuum, and washed with butanol (180 mL). Wet tadalafil crystals (28.9 g, 99.5% by HPLC assay) were obtained.

### Example 2

### Crystallization of Tadalafil in Acetone/Methanol

Crude tadalafil (20 g of dry base), acetone (600 mL), and methanol (120 mL) were combined to form a solution of crude tadalafil in a one liter reactor equipped with a mechanical stirrer, a condenser, and a thermometer. The solution was heated to 50° C. and stirred at a rate of 100 rpm for about one hour to obtain a clear solution. The solution was then filtered and cooled to about 20° C. and seeding was performed. The mixture was stirred at about 20° C. for one hour, and then cooled further to 0° C. over three hours. The mixture was stirred at 0° C. for an additional hour, and then filtered under vacuum, and washed with methanol (120 mL). Wet tadalafil crystals (12.4 g, 100% by HPLC assay) were obtained.

### Example 3

### Crystallization of Tadalafil in Acetone/Water

Crude tadalafil (30 g of dry base), acetone (900 mL), and water (90 mL) were combined to form a solution of crude tadalafil in a one liter reactor equipped with a mechanical stirrer, a condenser, and a thermometer. The solution was heated to 30° C. and stirred at a rate of 200 rpm for about one hour to obtain a clear solution. The solution was then cooled to about 12° C. and seeding was performed. The mixture was stirred at about 12° C. for one hour, and then cooled further to −10° C. over 12 hours. The mixture was stirred at −10° C. for an additional 6 hours, and then filtered under vacuum, and washed with methanol (120 mL). Wet tadalafil crystals (22.35 g, 99.7% by HPLC assay) were obtained. According to the XRD analysis of the obtained material, tadalafil Form I was obtained.

### Example 4

### Crystallization of Tadalafil in Acetonitrile/Water

Crude tadalafil (35 g of dry base), acetonitrile (680.5 mL), and water (160 mL) were combined to form a solution of crude tadalafil in a one liter reactor equipped with a mechanical stirrer, a condenser, and a thermometer. The solution was heated to a jacket temperature of about 85° C. and stirred at a rate of 150 rpm for about one hour until a clear solution was obtained and distillation had begun. The distillation continued until half of the volume of the solution remained. The temperature of the solution was about 77° C. The solution was stirred at this temperature for another two hours. The solution was then cooled to about 10° C. over about 3 hours, and then stirred at this temperature for another 13 hours. The solution was then filtered under vacuum and washed with acetonitrile (170 mL) and water (40 mL) to obtain wet tadalafil crystals (21.45 g, 99.9% by HPLC assay).

What is claimed is:

1. A process of purifying tadalafil comprising the steps of:
a) providing a solution of crude tadalafil in a solvent selected from the group consisting of C₂-C₆ aliphatic alcohols and mixtures of ketones or nitrites with a hydroxylic solvent; and
b) crystallizing purified tadalafil from the solution.

2. The process of claim 1, wherein the solvent is selected from the group consisting of: butanol, a mixture of acetone and methanol, and mixtures of acetone or acetonitrile with water.

3. The process of claim 2, wherein the solvent is a mixture of acetone and water.

4. The process of claim 1, wherein the solution contains about 15 to about 100 volumes of solvent per gram of crude tadalafil.

5. The process of claim 4, wherein the solution contains about 20 to about 70 volumes of solvent per gram of crude tadalafil.

6. The process of claim 1, wherein the solution is heated to a temperature of about 25° C. to about 140° C. prior to crystallization of tadalafil.

7. The process of claim 6, wherein the solution is heated to a temperature of about 50° C. to about 125° C.

8. The process of claim 7, wherein the solution is heated to a temperature of about 50° C. to about 95° C.

9. The process of claim 1, wherein the crystallizing of step b) is performed by cooling the solution to a temperature of about −20° C. to about 45° C.

10. The process of claim 9, wherein the crystallizing of step b) is performed by cooling the solution to a temperature of about 10° C. to about 35° C.

11. The process of claim 1, further comprising seeding the solution.

12. The process of claim 1, wherein the purified tadalafil contains about 0.5% area by HPLC or less of impurities.

13. The process of claim 12, wherein the purified tadalafil contains about 0.2% area by HPLC or less of impurities.

14. The process of claim 1, wherein the purified tadalafil has an assay of at least about 99.5% w/w.

15. The process of claim 14, wherein the purified tadalafil has an assay of about 100% w/w.

16. The process of claim 1, wherein the purified tadalafil contains about 20 ppm to about 300 ppm of methyl amine.

17. Tadalafil containing about 20 ppm to about 300 ppm of methyl amine.
